(19) United States
(12) Patent Application Publication
(10) Pub. No.: US 2020/0383878 A1
(43) Pub. Date: Dec. 10, 2020

(54) DENTURE BASE RESIN FOR 3D PRINTING

(71) Applicants: CYBERMED INC., Seoul (KR); WONKWANG UNIVERSITY CENTER FOR INDUSTRY-ACADEMY COOPERATION, Jeollabuk-do (KR)

(72) Inventors: Ji Myung BAE, Jeollabuk-do (KR); Da Ryeong PARK, Jeollabuk-do (KR); Seong Jin SHIN, Jeollabuk-do (KR)

(21) Appl. No.: 16/764,544

(22) PCT Filed: Nov. 19, 2018
(86) PCT No.: PCT/KR2018/014211

§ 371 Date: May 15, 2020

(30) Foreign Application Priority Data
Nov. 17, 2017 (KR) 10-2017-0154268
Dec. 1, 2017 (KR) 10-2017-0164015

(51) Int. Cl. A61K 6/887; A61C 13/01; A61K 6/893; B33Y 70/10

(52) U.S. Cl. CPC A61K 6/887; B33Y 70/10; A61K 6/893; A61C 13/01

(57) ABSTRACT

Disclosed is a denture base resin for 3D printing that comprises 30 wt %-43 wt % of urethane dimethacrylate (UDMA).

### FIELD

The present disclosure generally relates to denture base resins for 3D printing, and in particular, to denture base resins for 3D printing featuring improved mechanical properties.

### BACKGROUND

This section provides background information related to the present disclosure which is not necessarily prior art.

In general, a mixture of PMMA (polymethylmethacrylate) powder and MMA (methylmethacrylate) liquid is commonly used denture base material, and heat-curing (e.g., thermal polymerization) type and self-curing type denture resins have been used for a long time. Particularly, PMMA resin is utilized as the molding material in place of glass because of its excellent mechanical properties including high transparency or clarity (i.e. it transmits about 95% of visible light), superior esthetics and a relatively high glass transition temperature. Moreover, most denture bases have been made from PMMA resin for quite some time because PMMA resin is stable in normal oral environments and have physical properties suitable for intraoral adaptation. Typically, denture base resins are required to have high levels of impact resistance and transparency. However, PMMA resin has a low impact strength, and as a result, it is easily damaged by external force such as drop impact. In addition, due to low surface hardness and low abrasion resistance, PMMA resin surface is more susceptible to abrasion or scratches, losing some of its transparency or clarity.

As the market for 3D printing has significantly grown, it has also penetrated the dental industry. Up to date, 3D printing has found its application in temporary crowns, splints, surgical guides, etc., among other dentistry supplies, but not in a prosthesis or any device that should be retained for an extended period of time inside the mouth where salivation occurs, temperature variations are present as different kinds of foods are introduced and consumed, chewing pressure is continuously applied while eating, and abnormal force from teeth grinding or jaw clenching is also applied. Therefore, denture base materials must have high mechanical properties (e.g., strength, elastic modulus, toughness, fatigue strength) enough to stand those circumstances mentioned previously, and they should not be cytotoxic.

People wear dentures at least during the day, meaning that this type of prosthesis is continuously retained within the oral cavity. Therefore, dentures need to satisfy requirements of the mechanical properties described above. Since 3D printing produces a dental prosthesis by building up materials layer by layer, the resulting prosthesis is much inferior to those prostheses produced with traditional methods using heat-cured or self-cured denture base resins containing fillers which typically serves to enhance mechanical properties. Conventional 3D printing technologies do not utilize resins with filler. Hence, they can only try to increase the degree of polymerization of resin monomers to enhance the mechanical properties of a denture. An increase in the degree of polymerization can be achieved by carefully selecting a specific type and content of resin monomer, adding a crosslinking agent, and choosing the type and content of photoinitiator suitable for the optical wavelength range of a 3D printer being used.

The major component of a dental resin matrix is a dimethacrylate-based composite, which has been developed to reduce polymerization shrinkage while increasing the degree of polymerization and enhancing mechanical properties. Bisphenol A-glycidyl methacrylate (Bis-GMA) and urethane dimethacrylate (UDMA) are most commonly used components for restorative resins in dentistry. UDMA is favored over Bis-GMA because it undergoes less polymerization shrinkage and has lower viscosity. Because UDMA monomers do not contain a phenol ring in their structures, they exhibit high elasticity and toughness and may accelerate polymerization. On the other hand, Bis-GMA is highly viscous and contains many functional groups such that Bis-GMA is made suitable for use with TEGDMA as a diluent. Co-monomer pentaerythritol tetraacrylate and di(trimethylolpropane) tetraacrylate are utilized as multifunctional monomer: a cross-linking agent, reactive diluent, and chemical intermediate, which offering fast cure response and a high crosslink density upon curing.

There is only one type of denture base resin currently available for 3D printing, i.e. NextDent from Vertex Dental. Unfortunately, this resin has low mechanical properties and poor aesthetics. In the case of heat-cured or self-cured denture base resins, nylon fibers contained in the resins have successfully matched and imitated the oral blood vessels and provided satisfactory aesthetic effects. However, no such fibers are incorporated into denture base resins for 3D printing such that it is not possible to duplicate the oral blood vessels, resulting in a prosthesis with only one color. Moreover, the color of the NextDent resin itself changes over time, and therefore the color stability of a prosthesis made of the resin for 3D printing gets degraded. An appropriate viscosity as well as suitable strength may also be needed in duplication of details by 3D printing. Additionally, the amount of residual monomer, water absorption and solubility must also exceed regulations stipulated in International Organization for Standardization (ISO) 20795-1 Dentistry-Base Polymers-Part 1: Denture base polymers. For example, heat-curing type denture base resins should have a flexural strength of at least 65 MPa, and a flexural modulus of at least 2 GPa. Self-curing resins should have a flexural strength of at least 60 MPa and a flexural modulus of at least 1.5 GPa.

One of the biggest problems with the production of 3D printed dentures nowadays is that denture bases and artificial teeth cannot be printed together at the same time as artificial teeth require much higher mechanical properties and have totally different colors and transparencies from those of the denture base. With no current technologies available for printing a denture base and artificial teeth together at the same time, denture bases are separately printed and then bonded to already existing artificial teeth, or both a denture base and artificial teeth are separately printed and then glued to each other later. Even though ISO 20795-1 and ISO 22112 Dentistry-Artificial teeth for dental prosthesis standards have not specified an upper limit for the bond strength between denture base and artificial teeth, if an adhesive failure occurs that the artificial teeth and the denture base fall apart cleanly at the interface, it is considered to have failed. On the other hand, other modes of failure, such as a cohesive failure where a failure occurs only in an artificial tooth or in the denture base and a mixed failure where both the adhesive failure and the cohesive failure occur, are technically considered passing. In the future, there are expectations that new technologies would be developed to print artificial tooth and a denture base together at the same time.

Therefore, the present disclosure is directed to provide a new denture base resin for 3D printing that contains a specific type and content of monomer and of photoinitiator, and a controlled amount of pigment. As compared with commercially available denture base resins for 3D printing, the resin according to the disclosure can provide enhanced mechanical properties, cytotoxicity, and bond strength between the resin and artificial teeth, and a broader range of applications.

### SUMMARY

This section provides a general summary of the disclosure and is not a comprehensive disclosure of its full scope or all of its features.

According to one aspect of the present disclosure, there is provided a denture base resin for 3D printing that contains urethane dimethacrylate (UDMA) in an amount of 30-43%.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIGS. 1 and 2 show components of a denture base resin for 3D printing according to the present disclosure.

FIGS. 3A and 3B shows a test method for flexural strength and flexural modulus of denture base resins for 3D printing according to the present disclosure.

FIGS. 4A and 4B illustrates the shape of a specimen used for bond strength testing of denture base resins for 3D printing according to the present disclosure, and a test method therefor.

FIGS. 5 and 6 present viscosity measurements of denture base resins for 3D printing according to the present disclosure.

FIGS. 7, 8 and 9 present flexural strength and elasticity modulus measurements of denture base resins for 3D printing according to the present disclosure.

FIGS. 10, 11 and 12 present bond strength measurements of denture base resins for 3D printing according to the present disclosure.

FIG. 13 illustrates different modes of failure in denture base resins for 3D printing according to the present disclosure.

FIGS. 14 and 15 present cytotoxicity measurements of denture base resins for 3D printing according to the present disclosure.

### DETAILED DESCRIPTION

The present disclosure will now be described in detail with reference to the accompanying drawing(s).

Denture base resins for 3D printing according to the present disclosure were fabricated and tested for viscosity, flexural strength, flexural modulus, bond strength and cytotoxicity, in comparison with commercially available denture base resins for 3D printing.

Five different monomers that are commercially available, including urethane dimethacrylate (hereinafter, UDMA), bisphenol A glycidyl methacrylate (hereinafter, Bis-GMA), triethylene glycol dimethacrylate (hereinafter, TEGDMA), Pentaerythritol tetraacrylate (hereinafter, PETRA), and di(trimethylolpropane)-tetraacrylate (hereinafter, Di-TMPTA), were mixed. Diphenyl (2,4,6-trimethylbenzoyl) phosphine oxide (hereinafter, DTPO) and ethyl 4-(dimethylamino) benzoate (hereinafter, DMAB) were then added as a photoinitiator and a photosensitizer, respectively. Erythrosin B and titanium oxide (hereinafter, TiO₂) were used as pigments. The contents (in wt %) of UDMA and DTPO were continuously modified to obtain optimal flexural strength and flexural modulus values. A commercially available denture base resin for 3D printer NextDent (Base, Vertex Dental, Netherlands) was used as the control group. The viscosity of this monomer mixture was measured, and flexural strength, elastic modulus, bond strength, and cytotoxicity were also evaluated. Data were analyzed by one-way ANOVA (p=0.05).

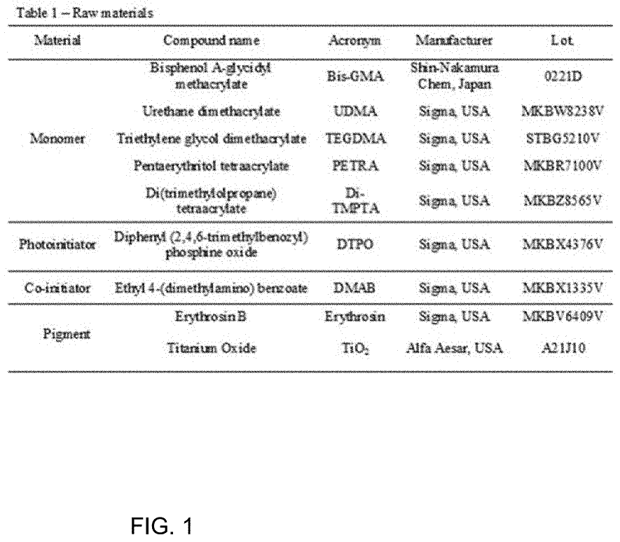

FIGS. 1 to 3 show components of a denture base resin for 3D printing according to the present disclosure.

The table in FIG. 1 lists materials of a denture base resin for 3D printing, component names, acronyms, and manufacturers of the components.

Those 3D printing denture resin bases for tests were obtained from the manufacturers listed in FIG. 1, and NextDent™ commercially available was selected for the control group (T0).

The monomers used for experiments include UDMA, Bis-GMA, TEGDMA), PETRA) and Di-TMPTA.

DTPO was used as a photoinitiator for experiments.

DMAB was used as a photosensitizer for experiments.

Erythrosin B and TiO₂ were used as pigments for experiments.

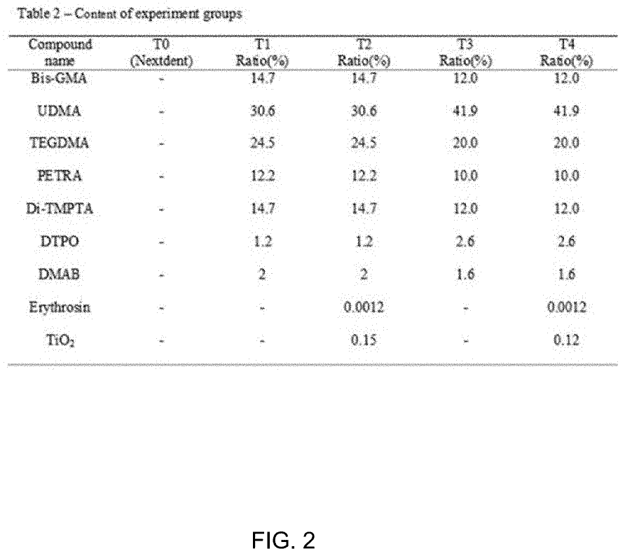

FIG. 2 compares test groups having different contents (in wt %) of the denture base resin for 3D printing according to the present disclosure, with the control group.

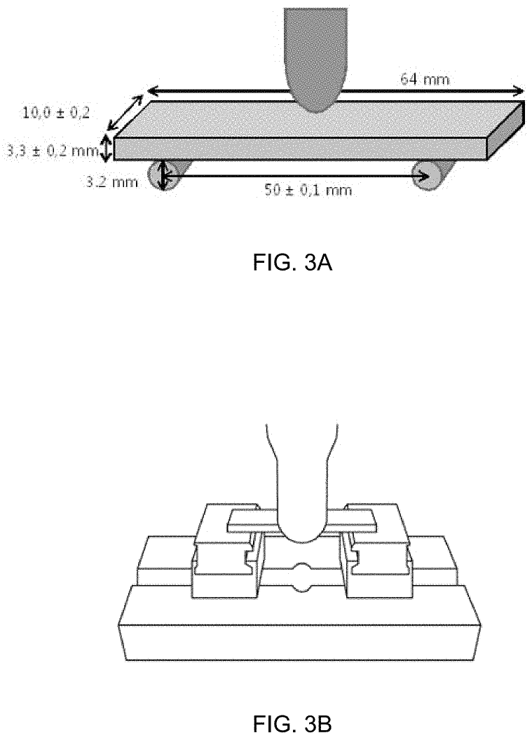

UDMA, Bis-GMA, TEGDMA, PETRA, and Di-TMPTA were mixed to obtain a monomer mixture. Test groups (T1-T4) were prepared with UDMA resin compound as a major component in the concentration of 30.6 wt % (T1 and T2) or 41.9 wt % (T3 and T4). In addition, DTPO (1.2 wt % or 2.6 wt %) and optionally Erythrosin (0.15 wt %) were added as a photoinitiator and a pigment, respectively. Also, TiO₂ (325 mesh) was added in an amount of 0.0012 wt % to provide opacity to the resin. In short, these four test groups T1-T4 have two different compositions, and two of them T2 and T4 contain pigments additionally (see FIG. 3). Listed below is the composition of each test group (every % here indicates percentage by weight).

Test group T1: Bis-GMA 14.7%, UDMA 30.6%, TEGDMA 24.5%, PETRA 12.2%, Di-TMPTA 14.7%, DTPO 1.2%, and DMAB 2%.

Test group T2: Bis-GMA 14.7%, UDMA 30.6%, TEGDMA 24.5%, PETRA 12.2%, Di-TMPTA 14.7%, DTPO 1.2%, DMAB 2%, Erythrosin 0.15%, and TiO₂ 0.0012%.

Test group T3: Bis-GMA 12.0%, UDMA 41.9%, TEGDMA 20.0%, PETRA 10.0%, Di-TMPTA 12.0%, DTPO 2.6%, and DMAB 1.6%.

Test group T4: Bis-GMA 12.0%, UDMA 41.9%, TEGDMA 20.0%, PETRA 10.0%, Di-TMPTA 12.0%, DTPO 2.6%, DMAB 1.6%, Erythrosin 0.15%, and TiO₂ 0.0012%.

In addition to the compositions in the test groups T1 and T3, the pigments Erythrosin and TiO₂ are added in the test groups T2 and T4 in order to match the gingival color.

To prepare specimens with a homogeneous mixture free of air bubbles, each test group was placed in a beaker on the stirrer with heating (RCH-3, Tokyo Rikakikai Co., LTD., Tokyo, Japan) set at 40° C. and mixed at the speed of 240 rpm for 1 hour by an overhead stirrer (RW20DZM.n, IKA-WERKE GmbH & Co.KG, Breisgau, Germany).

Measurements of viscosity (n, Pa s) were then performed on every test group with a viscometer (DV2TRVTJ0, No. 8692529, Brookfield Ametek, USA) and #21 spindle at 25° C. and at a speed of 60%-90% Torque.

FIG. 3 shows a test method for flexural strength and flexural modulus of denture base resins for 3D printing according to the present disclosure.

FIG. 3A is a schematic view of a flexure test to measure flexural strength and flexural modulus, and FIG. 3B is a photograph showing how the flexure test is carried out.

All resins were subjected to 3D printing through the mask image projection and resin curing process. The resulting specimens were cut in rectangular solid shape (64 mm×10 mm×3.3 mm) for the measurement of flexural strength and flexural modules. After 3D printing, all specimens were post-cured for 20 min with UV blue light box Digital Light Processing (UV; LC-3DPrint®, NextDent, Soesterberg, Netherlands), immersed in water and put in an oven (FO-600M, JEIO TECH, Korea) at 37° C. for 24 hours.

Flexural strength of the specimen was measured according to ISO 20795-1[17], using a universal tester (Z020, Zwick, Germany) with the crosshead speed of 5 mm/min, until failure. Elasticity modulus (E, GPa) was then calculated from the data obtained from the initial linear portion of the load-displacement curve. σ and E were calculated from Eq. (1) and Eq. (2) below.

$$\begin{matrix}σ = \frac{3 FL}{2 {bh}^{2}} , and & ( 1 ) \\ E = \frac{{F}_{1} {L}^{3}}{4 {bh}^{3} d} & ( 2 )\end{matrix}$$

wherein F denotes a maximum load (MPa); Fi denotes the load (N) at a selected point of the elastic region on the load-displacement curve; L denotes a distance between the supports (50 mm); b and h denote respectively the width and thickness of a specimen measured immediately before the specimen is immersed in water; and d denotes the deflection of the specimen under the load F₁

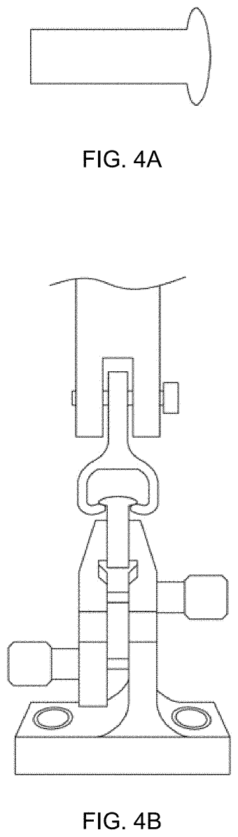

FIG. 4 shows a specimen used for bond strength testing of denture base resins for 3D printing according to the present disclosure, and a test method therefor.

FIG. 4A is a photograph of the specimen prepared for the bond strength test, and FIG. 4B is a photograph showing that the specimen is mounted on a bond strength testing jig produced according to ISO 22112:2005.

Specimens for bond strength testing were prepared according to ISO 22112:2005. Six anterior artificial teeth from maxillary left and right central incisor, lateral incisor and canine (Biotone, Dentsply, USA) were used. For the test, a total of 300 specimens were prepared in 5 groups, including the control group T0 using any commercially available denture base resin as in the flexural strength test, and four test groups T1, T2, T3 and T4. Specimen preparation was performed by scanning the ridge lap region of an artificial tooth, followed by 3D printing of a denture base resin based on the scan, in dimensions of 20 (L)×6.2 (W)×6.2 (D) mm. The interface area between the 3D printed denture base resin and the artificial teeth and the ridge lap area of the artificial teeth were abraded by 50 μm Al₂O₃ particles (Aluminum oxide, Danville, Germany) for 30 seconds at 2 bar air pressure to increase their adherence. All the specimens were then subjected to ultrasonic cleaning in distilled water at a frequency of 40 kHz for 20 minutes to remove any residual particles. Next, the specimens were dried at room temperature. Self-adhesive resin cement (Rely X™ U200, 3M ESPE, Deutschland) was utilized to bond the artificial teeth to 3D printed denture base resin patterns. While keeping the artificial teeth bonded to the artificial teeth under pressure of a static loading device, all the surfaces were photopolymerized for 40 seconds using an LED photo-polymerizer (VALO, Ultradent, USA). In order to apply a constant pressure, a load of 2 kg was placed on top of the static loading device. After 24 hours, the specimens were connected to a bond strength testing jig proposed in ISO 22112: 2005 and tested for bond strength using a universal tester with the crosshead speed of 5 mm/min, until failure.

For cytotoxicity testing, specimens in each group were prepared in dimensions of 10 (L)×10 (W)×3.3 (D) mm. According to ISO10993-5 (Biological evaluation of medical devices-Part 5: Tests for in vitro cytotoxicity), the specimens were placed in 24-well plates with RPMI medium and put in a 37° C. oven for 24 hours for extraction. The extraction rate was set such that the ratio of the surface area of a specimen to the extraction solution would be 3 cm²/mL, as defined in ISO10993-12 (Biological evaluation of medical devices-Part 12: Sample preparation and reference materials). An aluminum oxide ceramic rod was used as a negative control, and 1% phenol was used as a positive control.

In this study, L929 cells (NCTC clone 929, CCL 1, ARCC) were used. RPMI medium (AB10131148, Hyclone, USA) containing 10% fetal bovine serum (FBS, Gibco) was cultured in a 37. 5% carbon dioxide incubator. Into a 96-well plate, 0.1 ml of the RPMI medium was dispensed up to 1×10⁴ cells/well and cultured for 24 hours. The culture medium was removed from each well, and 100 μl of the extract and RPMI medium of each resin group was added at 37° C. for 24 hours. 50 μl of 1 mg/ml MTT solution (3-(4,5-Dimethylthiazol-2-yl)-2,5-Diphenyl tetrazolium Bromide; Thiazolyl Blue Tetrazolium Bromide, Sigma, USA) was added to each well. In order to protect the cells from damage, the plate was covered with aluminum foil and placed in an oven at 37° C. for 3 hours. Absorbance was measured at a wavelength of 570 nm on an ELISA reader (Spectra max 250, molecular devices, USA). The test was repeated three times independently for each group.

The measurements obtained were analyzed at a significance level p<0.05 using one-way ANOVA (Analysis of variance) and post hoc Tukey's HSD (Honestly Significant Difference) pairwise multiple comparisons. All calculations were carried out by IBM SPSS Statistic 22 software (SPSS Inc., Chicago, Ill., USA).

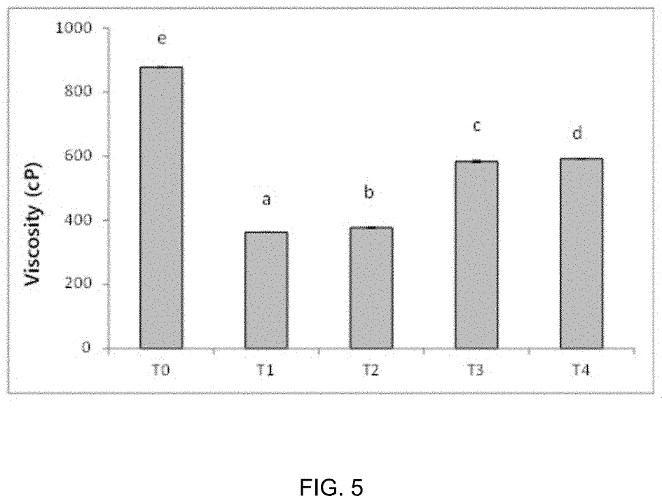

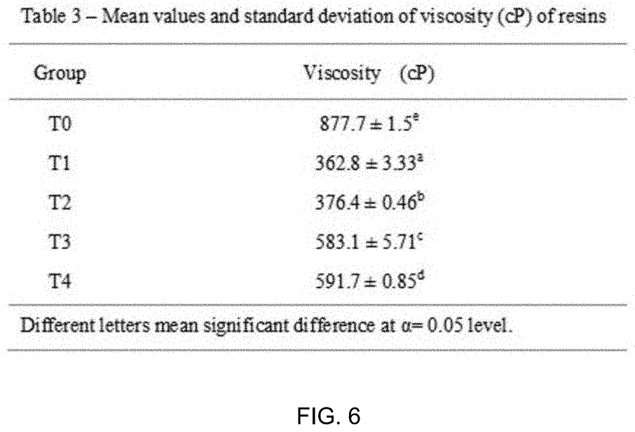

FIGS. 5 and 6 present viscosity measurements of denture base resins for 3D printing according to the present disclosure.

FIG. 5 lists means and standard deviations of the viscosity measurements in the test groups. It was observed that all differences between the groups were statistically significant (p<0.05). The control group T0 was twice more viscous than the other test groups and showed the highest mean value. The viscosity of the UDMA-based groups continued to increase as the concentration of UDMA increases. Further, the test groups T2 and T4, each containing the pigments Erythrosin and TiO₂ showed higher viscosities than the test groups T1 and T3.

It turned out that the viscosity of the control group T0 (NextDent) was the highest, and the viscosity of the test group T1 among others was significantly lowest (p<0.05). Again, the test groups T2 and T4, to which the pigments Erythrosin and TiO₂ were added, showed higher viscosities than the test groups T1 and T3. It is understood that the presence of pigment(s) brings a change in viscosity, and that the viscosity increases as the content of UDMA increases.

Since 3D printers create tangible objects by building up materials consecutively layer by layer of constant thickness, the viscosity of a resin used for 3D printing has a great impact on the printing result. It is said that materials of high viscosity tend to produce more slurries after polymerization. The control group T0 showed a viscosity (877.7±1.5) of about three times higher than the prepared denture base resins for 3D printing in the test groups T1-T4, such that their specimens have better fluidity than the specimen of the control group T0. Moreover, during the 3D printing process, the denture base resins for 3D printing in the test groups T2 and T4 produce less slurries than in the test groups T1 and T3, such that a smoother surface can be obtained, and detailed parts are reproduced better.

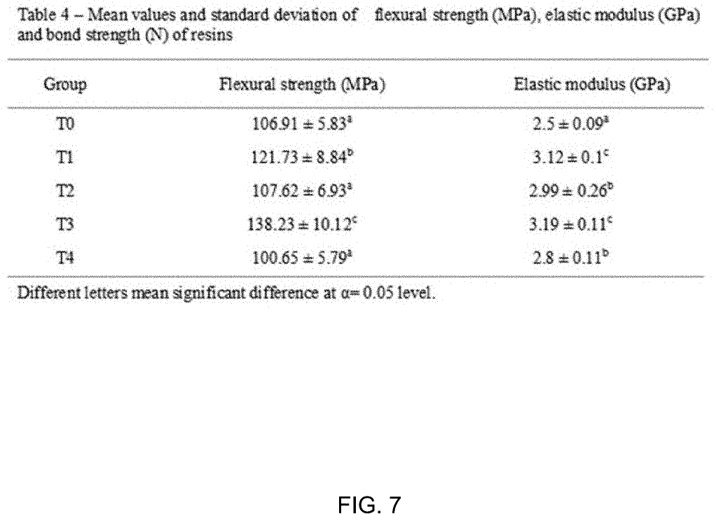

FIGS. 7 to 9 present flexural strength and elasticity modulus measurements of denture base resins for 3D printing according to the present disclosure.

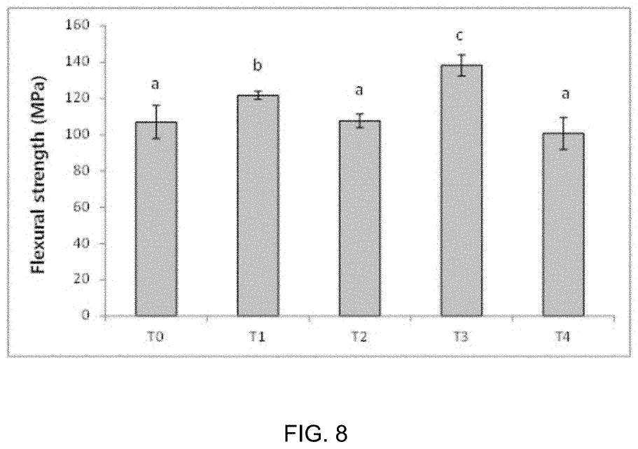

Results from the flexural strength test were inverse to results from the viscosity test (see FIG. 8). The test group T3 containing 41.9 wt % of UDMA demonstrated the highest flexural strength of 138.23 MPa (p<0.05), and the test group T1 containing 30.6 wt % of UDMA demonstrated the second highest flexural strength of 121. 71 MPa (p<0.05). Hence, the resin with a larger UDMA content can demonstrate a higher flexural strength.

Meanwhile, the test groups T2 and T4 containing the pigments Erythrosin and TiO₂ showed substantially lower flexural strengths, 107.62 MPa and 100.65 MPa, respectively. However, there was no significant difference between these two groups (p>0.05).

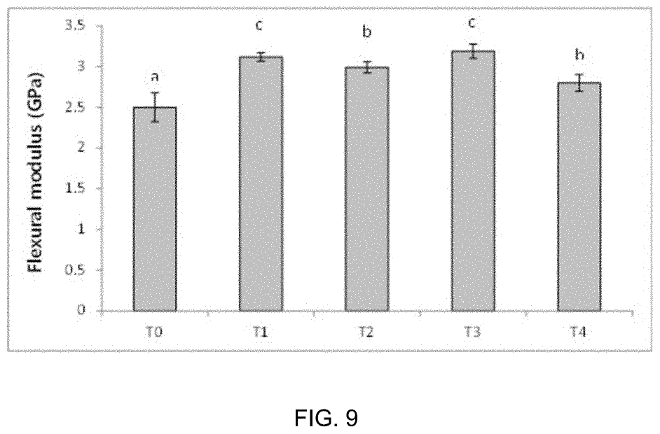

Referring next to FIG. 9, the groups that demonstrated high flexural strength also had high flexural modulus. For example, higher flexural modulus values (p<0.05) were found in the test groups T1 and T3 free of the pigments Erythrosin and TiO₂, but there was no significant difference between these two groups (p>0.05). The test groups T2 and T4 containing the pigments Erythrosin and TiO₂, on the other hand, had lower flexural modulus values (p<0.05), but there was no significant difference between these two groups (p>0.05). In addition, the flexural modulus of the control group T0 was significantly lowest among all the groups (p<0.05).

As described previously, the test groups T2 and T4 to which the pigments Erythrosin and TiO₂ were added had lower flexural strength and flexural modulus than the test groups T1 and T3 without the pigments. It is believed that when the pigments Erythrosin and TiO₂ are incorporated into a denture base resin, the resin gets darker and less transparent due to the pigment particles and would have a lower degree of polymerization in a digital light processing (DLP) 3D printer, for example, resulting in a decrease in flexural strength and flexural modulus.

ISO 20795-1: 2008 stipulates requirements to be met: for example, the ultimate flexural strength of heat-polymerized resins for denture bases shall be at least 65 MPa, the ultimate flexural strength of self-cured resins shall be at least 60 MPa, the elastic modulus of heat-polymerized resins shall be at least 2 GPa, and the elastic modulus of self-cured resins shall be at least 1.5 GPa. All the test groups T1-T4 according to the present disclosure satisfied the ISO requirements of flexural strength and elastic modulus for heat-polymerized resins. In particular, the flexural strength of the test group T3 containing 41.19 wt % of UDMA was the highest value (138.23±10.12 MPa) (p<0.05). In addition, a higher modulus of elasticity was found in the test group T1 (3.12±0.1 GPa) and the test group T3 (3.19±0.11 GPa) (p<0.05). Thus, it can be concluded that the flexural strength and elastic modulus increase as the content of UDMA increases, whereas the flexural strength and elastic modulus decrease when pigments are present in the resin.

In short, the test groups T1 and T3 demonstrated significantly higher flexural strength than the control group T0, and all the test groups T1-T4 had a higher flexural modulus than the control group (T0). After all, each of the test groups demonstrated flexural strength of at least 65 MPa and flexural modulus of at least 2 GPa, as required by ISO standards.

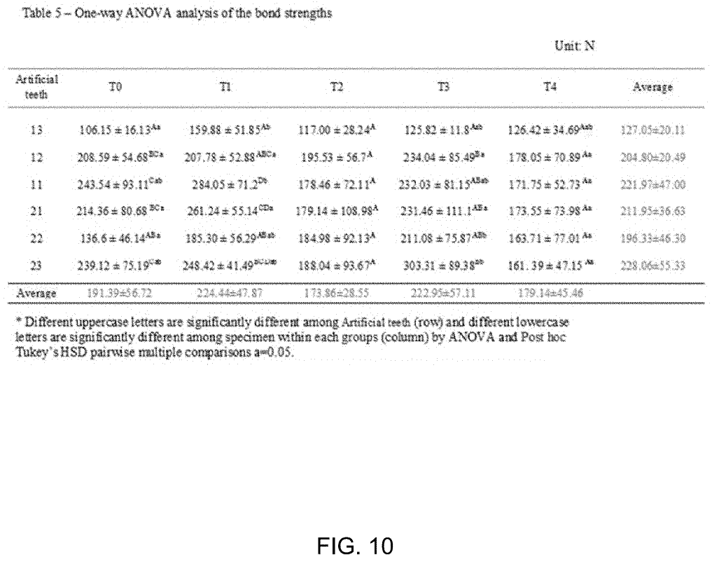

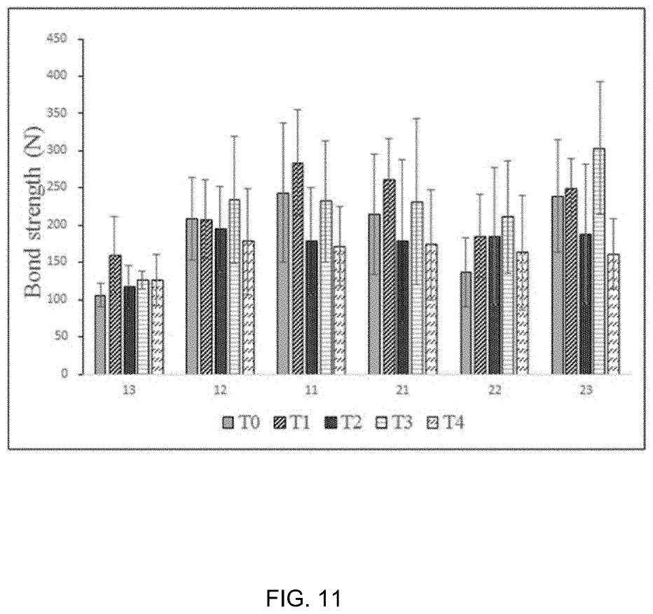

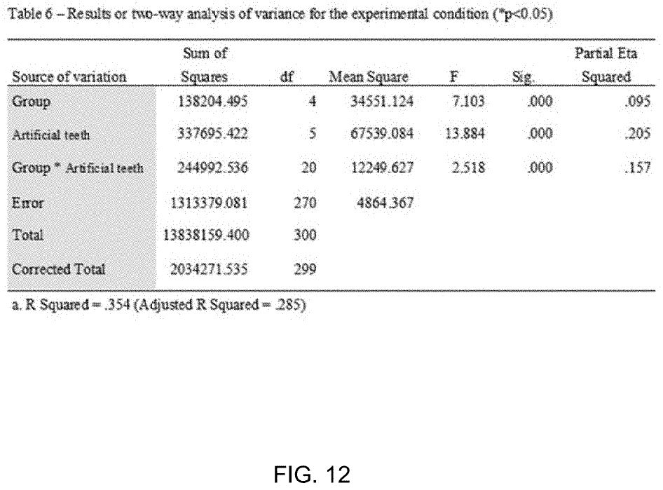

FIGS. 10 to 12 present bond strength measurements of denture base resins for 3D printing according to the present disclosure.

The comparison result of the mean value of bond strengths of six artificial anterior teeth for each test revealed that the bond strength was significantly higher in the test groups T1 and T3 than in the other test groups (p<0.05). It is believed that the pigments Erythrosin and TiO₂ not only affect the strength itself, but they also affect the bonding to cement, causing deterioration in the overall bond strength. Also, there were significant differences among the artificial teeth (depending on which of the six anterior teeth) (p<0.05). With different artificial teeth, tooth No. 23 demonstrated the highest bond strength (303.31±89.38 N). This implies that tooth size might have an impact on the bond strength. In effect, when artificial teeth and a 3D printed resin were cemented, teeth having a relatively larger surface area tended to have higher bond strengths.

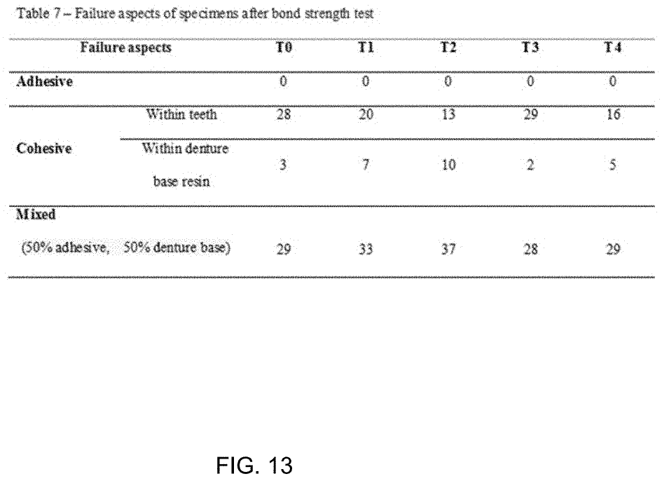

FIG. 13 illustrates different modes of failure in denture base resins for 3D printing according to the present disclosure.

As can be seen in FIG. 13, specimens in every test group showed two modes of failure: cohesive failure and mixed failure. In particular, the cohesive failure occurred as a fracture was observed in the artificial teeth as well as in the denture base resins of the test groups. ISO 22112:2009 stipulates that among the failure modes, cohesive or mixed failure, not adhesive failure, should occur in the artificial tooth or denture base resin to be technically considered to have passed. The mixed failure refers to a case where any residual resin remains adhered to the artificial teeth, or artificial tooth remnants remain adhered to the resin. According to ISO 22112 standards, specimens with only 100% adhesive failure are technically considered to have failed, but none of the specimen in the test groups fell into that category. Since artificial teeth are cemented to a 3D printed denture base resin, it is important to find out which failure mode occurred. When the adhesion between an artificial tooth and a denture base resin is high, either the cohesive or mixed failure occurs in the tooth or resin; when the adhesion is low, the adhesion failure occurs at the interface between the tooth and the denture base resin.

Referring back to FIG. 13, all of the test groups T1-T4 showed the cohesive and/or mixed failure, implying that they successfully met ISO standards.

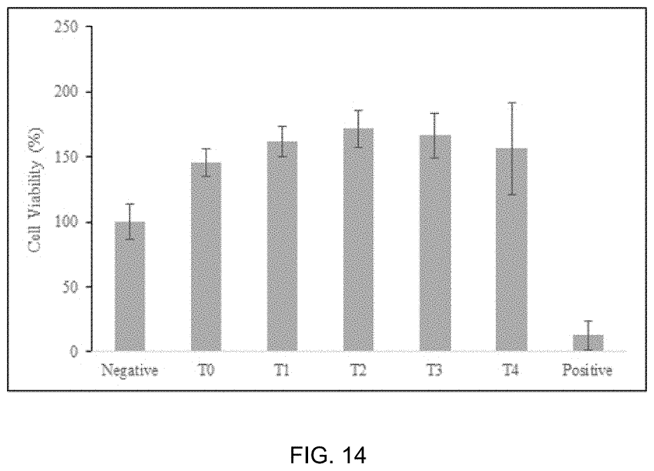

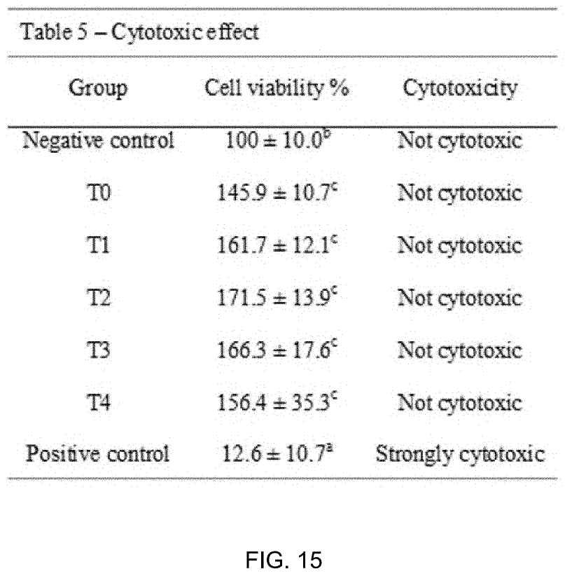

FIGS. 14 and 15 present cytotoxicity measurements of denture base resins for 3D printing according to the present disclosure.

All the prepared resins were eluted for 24 hours and cell activity measurements were obtained as shown in FIG. 14. Every test group demonstrated higher cell activity than the negative control (p<0.05). It is stated in ISO 10993-5: 2009 (E) that materials are free of cytotoxicity if the cell activity of the materials is at least 70% according to MTT assay results. Every test group used in this experiment showed cell activity that is not only 70% or more, but also higher than that of the negative control. This confirmed that the test groups are free of cytotoxicity and have biocompatibility.

Therefore, it can be concluded that all of the test groups according to the disclosure are clinically applicable.

Listed below is a range of % (by weight) for each compound in the denture base resin of each test group T1-T4.

Test group T1: Bis-GMA 14.4%-15%, UDMA 30.2%-30.9%, TEGDMA 24.2%-25%, PETRA 10.2%-12.5%, Di-TMPTA 14.7%-15%, DTPO 0.5%-2%, and DMAB 1.6%-2.1%.

Test group T2: Bis-GMA 14.4%-15%, UDMA 30.2%-30.9%, TEGDMA 24.2%-25%, PETRA 10.2%-12.5%, Di-TMPTA 14.7%-15%, DTPO 0.5%-2.7%, DMAB 1.6%-2.1%, Erythrosin 0.0012%-0.006%, and TiO₂ 0.12%-0.15%.

Test group T3: Bis-GMA 11.8%-12.2%, UDMA 41.3%-43%, TEGDMA 19.7%-20.4%, PETRA 9.8%-10.2%, Di-TMPTA 11.8%-12.2%, DTPO 0.4%-4%, and DMAB 1.6%-2.1%.

Test group T4: Bis-GMA 11.8%-12.2%, UDMA 41.3%-43%, TEGDMA 19.7%-20.4%, PETRA 9.8%-10.2%, Di-TMPTA 11.8-12.2%, DTPO 0.4%-4%, DMAB 1.6%-2.1%, Erythrosin 0.0012%-0.006%, and TiO₂ 0.12%-0.15%.

The present disclosure is designed to provide denture base resins suitable for 3D printing in any 3D printer, and to evaluate the mechanical and biological properties of the resins.

As a result of evaluation, it was found that the 3D printed denture base resins according to the present disclosure satisfied requirements of the mechanical and biological properties as stated in ISO standards. In particular, the test group T3 turned out to be superior to the control group T0, a commercially available denture base resin for 3D printing, in all the areas including flexural strength, elasticity modulus, bond strength, and MTT test measurements.

There is still a need for developing denture base resin materials suitable for 3D printing that can reproduce the actual colors and textures of teeth and gingiva as much as possible, through modifications of the amounts of pigments and opacity particles to be added to the resin materials.

The following describes evaluation results of the mechanical and biological properties of denture base resins suitable for 3D printing in any 3D printer according to the present disclosure.

Among others, denture resins for 3D printing in the test group T3 demonstrated statically significantly highest values of flexural strength and elastic modulus (p<0.05).

MTT test results also confirmed that all of the test groups had cytotoxicity of 70% or less.

As compared with commercially available denture base resins for 3D printing, those denture base resins in the test group T3 according to the present disclosure showed excellent mechanical properties, and their biological properties successfully met ISO standards.

In particular, the test groups T1 and T3 had lower viscosity and higher flexural strength and elastic modulus than the control group T0.

All parameters were determined based on UDMA and DPTO content. For example, the viscosity of each test group continued to increase as the concentration of UDMA increases, and the presence of pigments also created a significant difference (p<0.05). The flexural strength, elasticity modulus, and bond strength of each resin were higher prior to the addition of pigments (p<0.05), and cytotoxicity was not found in the resins (p>0.05). Once pigments were added, however, there were significant differences in flexural strength and elastic modulus (p<0.05).

It was confirmed that the pigments affected the mechanical properties of the denture base resins for use in 3D printers. In addition, the inventors learned that a combination of an adequate increase in the content of non-cytotoxic UDMA monomer and incorporation of the photoinitiator DTPO also provided excellent properties to the resins.

DTPO is the most widely used photoinitiator for 3D printers as it is known to have an optical wavelength band closest to most 3D printers used in the dental industry.

Set out below are a series of clauses that disclose features of further exemplary embodiments of the present disclosure, which may be claimed.

(1) A denture base resin for 3D printing, comprising: bisphenol A-glycidyl methacrylate (Bis-GMA), urethane dimethacrylate (UDMA), triethylene glycol dimethacrylate (TEG DMA), pentaerythritol tetraacrylate (PETRA), and Di(trimethyllopropane)-tetraacrylate (Di-TMPTA).

(2) There is also provided, the denture base resin for 3D printing of clause (1) wherein: the resin comprises 12 wt %-15 wt % of Bis-GMA, 0 wt %-31 wt % of UDMA, 20 wt %-25 wt % of TEGDMA, 10 wt %-13 wt % of PETRA, and 12 wt %-15 wt % of Di-TM PTA.

(4) There is also provided, the denture base resin for 3D printing of clause (1) further comprising: a photoinitiator.

(5) There is also provided, the denture base resin for 3D printing of clause (3) wherein: the photoinitiator is DTPO.

(6) There is also provided, the denture base resin for 3D printing of clause (1) further comprising: 0 wt %-1.2 wt % of DTPO.

(7) There is also provided, the denture base resin for 3D printing of clause (1) further comprising: 1.2 wt %-3 wt % of DTPO.

(8) There is also provided, the denture base resin for 3D printing of clause (1) further comprising: a photosensitizer.

(9) There is also provided, the denture base resin for 3D printing of clause (7) wherein: the photosensitizer is DMAB.

(10) There is also provided, the denture base resin for 3D printing of clause (1) further comprising: 0 wt %-1.6 wt % of DMAB.

(11) There is also provided, the denture base resin for 3D printing of clause (1) further comprising: 1.6 wt %-2 wt % of DMAB.

(12) There is also provided, the denture base resin for 3D printing of clause (1) further comprising: pigments.

(13) There is also provided, the denture base resin for 3D printing of clause (11) wherein: the pigments include Erythrosin and TiO₂.

(14) There is also provided, the denture base resin for 3D printing of clause (1) further comprising: 0 wt %-0.0012 wt % of Erythrosin and 0.12 wt %-0.2 wt % of TiO₂.

(15) There is also provided, the denture base resin for 3D printing of clause (1) further comprising: 0 wt %-0.0012 wt % of Erythrosin and 0 wt %-0.12 wt % of TiO₂.

(16) There is also provided, the denture base resin for 3D printing of clause (1) wherein: UDMA is included in an amount of 30 wt %-43 wt %.

If the content of UDMA falls below 30 wt %, the denture base resin for 3D printing could have lower strength. Similarly, if the content of UDMA is above 43 wt %, the strength of the denture base resin for 3D printing could be reduced. The resin demonstrated the highest strength when the content of UDMA is between 41.3 wt % and 43 wt %.

(17) There is also provided, the denture base resin for 3D printing of clause (1) wherein: the resin comprises 30.2 wt %-30.9 wt % of UDMA and 0.5 wt %-2.6 wt % of DTPO.

If the content of UDMA falls below 30.2 wt % or goes above 30.9 wt %, the denture base resin for 3D printing could have lower strength. Meanwhile, the content of UDMA between 30.2 wt % and 30.9 wt % provides adequate viscosity, such that the resin would have a smoother surface and demonstrate high strength.

In addition, if the content of DTPO falls below 0.2 wt %, the degree of polymerization is rather low. Meanwhile, if the content of DTPO is above 2.6 wt %, the degree of polymerization gets so high that a fully shaped 3D printed object may not even obtained due to such overpolymerization in advance. Therefore, the optimal range of the DTPO content falls between 0.2 wt % and 2.6 wt % to achieve best polymerization.

(18) There is also provided, the denture base resin for 3D printing of clause (1) wherein: the resin comprises 30.2 wt %-30.9 wt % of UDMA, 0.5 wt %-2.6 wt % of DTPO, 0.0012 wt %-0.006 wt % of Erythrosin, and 0.12 wt %-0.15 wt % of TiO₂.

Again, the denture base resin for 3D printing could have lower strength if the content of UDMA falls below 30.2 wt % or goes above 30.9 wt %. Meanwhile, the content of UDMA between 30.2 wt % and 30.9 wt % can provide adequate viscosity, such that the resin would have a smoother surface and demonstrate high strength.

The degree of polymerization is rather low if the content of DTPO falls below 0.2 wt %. However, as mentioned previously, if the content of DTPO is above 2.6 wt %, the degree of polymerization gets so high that a fully shaped 3D printed object may not even obtained due to such overpolymerization in advance. Therefore, the optimal range of the DTPO content falls between 0.2 wt % and 2.6 wt % to achieve excellent polymerization.

Moreover, if Erythrosin is included in an amount less than 0.0012 wt %, the resulting color shall not be aesthetically pleasing. If it is included in an amount greater than 0.006 wt %, however, the resulting color might turn out to be too red. Besides, an unnecessarily high content of pigments is not desirable because the degree of polymerization can decrease, and the strength may decrease as well. Erythrosin reproduces the most natural color when its content is between 0.0012 wt % and 0.006 wt %.

If TiO₂ is included in an amount less than 0.12 wt %, the resin would be transparent instead of being sufficiently opaque, making it aesthetically unpleasing. If TiO₂ is included in an amount greater than 0.15 wt %, it means that the resin will have an increased amount of particles, resulting in undesirable consequences such as poor strength, a high degree of opacity and unappealing aesthetics.

(19) There is also provided, the denture base resin for 3D printing of clause (1) wherein: the resin comprises 41.3 wt %-43 wt % of UDMA and 0.4 wt %-4 wt % of DTPO.

If the content of UDMA falls below 41.3 wt % or goes above 43 wt %, the denture base resin for 3D printing could have lower strength. Meanwhile, the content of UDMA between 41.3 wt % and 43 wt % can provide adequate viscosity and fluidity, which in turn leads to highly accurate printing performances.

In addition, if the content of DTPO falls below 0.4 wt %, the degree of polymerization is rather low. Meanwhile, if the content of DTPO is above 4 wt %, the degree of polymerization gets so high that a fully shaped 3D printed object may not even obtained due to such overpolymerization in advance. Therefore, the optimal range of the DTPO content falls between 0.4 wt % and 4 wt % to achieve a proper level of polymerization.

(20) There is also provided, the denture base resin for 3D printing of clause (1) wherein: the resin comprises 41.3 wt %-43 wt % of UDMA, 0.4 wt %-4 wt % of DTPO, 0.0012 wt %-0.006 wt % of Erythrosin, and 0.12 wt %-0.15 wt % of TiO₂.

If the content of UDMA falls below 41.3 wt % or goes above 43 wt %, the denture base resin for 3D printing could have lower strength. Meanwhile, the content of UDMA between 41.3 wt % and 43 wt % can provide adequate viscosity and fluidity, which in turn leads to highly accurate printing performances.

In addition, if the content of DTPO falls below 0.4 wt %, the degree of polymerization is rather low. Meanwhile, if the content of DTPO is above 4 wt %, the degree of polymerization gets so high that a fully shaped 3D printed object may not even obtained due to such overpolymerization in advance. Therefore, the optimal range of the DTPO content falls between 0.4 wt % and 4 wt % to achieve a proper level of polymerization. Moreover, if Erythrosin is included in an amount less than 0.0012 wt %, the resulting color shall not be aesthetically pleasing. If it is included in an amount greater than 0.006 wt %, however, the resulting color might turn out to be too red. An increased among of particles may also decrease the strength. Erythrosin reproduces the most natural color when its content is between 0.0012 wt % and 0.006 wt %.

Further, if TiO₂ is included in an amount less than 0.12 wt %, the resin would be transparent instead of being sufficiently opaque, making it aesthetically unpleasing. If TiO₂ is included in an amount greater than 0.15 wt %, it means that the resin will have an increased amount of particles, resulting in undesirable consequences such as poor strength, a high degree of opacity and unappealing aesthetics.

(21) There is also provided, the denture base resin for 3D printing of clause (1) wherein: the resin comprises 1.6 wt %-2.1 wt % of DMAB.

If DMAB is included in an amount below 0.16 wt %, it will not properly function as a photosensitizer, and the degree of polymerization may be lowered. Meanwhile, if DMAB is included in an amount above 2.1 wt %, excess absorption of light occurs, and thus light curing occurs to a greater extent. Therefore, together with a photoinitiator, DMAB in an amount between 1.6 wt % and 2.1 wt % can provide a proper level of polymerization.

(22) There is also provided, the denture base resin for 3D printing of clause (1) wherein: the resin comprises 19.2 wt %-25 wt % of TEGDMA.

If the content of TEGDMA falls below 19.2 wt %, the denture base resin for 3D printing could have lower fluidity such that the components of the resin would not mix well together. Meanwhile, if the content of TEGDMA which is a diluent is above 25 wt %, the resin is diluted due to excess amount of the diluent and the strength of the resin is reduced. Therefore, the optimal range of the TEGDMA content falls between 19.2 wt % and 25 wt % to achieve sufficient fluidity and better mixing behavior of all materials of the resin.

(23) There is also provided, the denture base resin for 3D printing of clause (1) wherein: the resin comprises 11.5 wt %-15 wt % of Bis-GMA.

If Bis-GMA is included in an amount below 15 wt %, the denture base resin for 3D printing could have lower strength. Meanwhile, if Bis-GMA is included in an amount above 19.2 wt %, the resin could be too viscous, causing many problems during the 3D printing process. Therefore, the optimal range of the Bis-GMA content falls between 15 wt % and 19.2 wt % to achieve adequate viscosity and high strength for the resin.

(24) There is also provided, the denture base resin for 3D printing of clause (1) wherein: the resin comprises 10 wt %-14.5 wt % of PENTRA.

If PENTRA is included in an amount below 10 wt %, the denture base resin for 3D printing could have lower strength. Meanwhile, if PENTRA is included in an amount above 14.5 wt %, the resin could be too viscous, adversely affecting 3D printing performance. Therefore, the optimal range of the PENTRA content falls between 10 wt % and 14.5 wt % to achieve adequate viscosity and adequate strength for the resin during the 3D printing process.

(25) There is also provided, the denture base resin for 3D printing of clause (1) wherein: the resin comprises 11.5 wt %-15 wt % of Di-TMPTA.

If Di-TMPTA is included in an amount below 11.5 wt %, the denture base resin for 3D printing could have lower strength. Meanwhile, if Di-TMPTA is included in an amount above 15 wt %, the resin could be too viscous, adversely affecting 3D printing performance. Therefore, the optimal range of the Di-TMPTA content falls between 11.5 wt % and 15 wt % to achieve adequate viscosity and adequate strength for the resin during the 3D printing process.

An exemplary denture base resin for 3D printing according to the present disclosure can be used in 3D printers.

An exemplary denture base resin for 3D printing according to the present disclosure satisfies requirements of ISO 20795-1 standards and is non-toxic.

An exemplary denture base resin for 3D printing according to the present disclosure is excellent in all the areas including flexural strength, elasticity modulus, bond strength, and MTT test measurements.

An exemplary denture base resin for 3D printing according to the present disclosure has a lower viscosity than the conventional materials, producing less slurries and forming a smooth surface.

An exemplary denture base resin for 3D printing according to the present disclosure shows cytotoxicity of not greater than 70%.

The comparison of denture base resins for 3D printing in test groups according to the present disclosure confirmed that there was a significant difference in the bond strength between the test groups T1 and T3 and the other test groups T2 and T4, and that all artificial teeth except for tooth No. 12 and tooth No. 21 in the test groups T1-T4 had a significant difference (p<0.05) in their bond strengths. In particular, tooth No. 23 in the test group T3 demonstrated the highest bond strength (303.31±89.38 N) (p<0.05). After observing failure modes in specimens, it turned out that all the test groups T1-T4 showed a cohesive failure and a mixed failure.

As compared with commercially available denture base resins for 3D printing, the denture base resins for 3D printing according to the present disclosure in the test groups T1 and T3 demonstrated excellent flexural strength and flexural modulus, lower viscosity, and higher bond strength to artificial teeth. Biological properties of those resins also satisfied requirements of ISO standards.

What is claimed is:

1. A denture base resin for 3D printing, comprising: 30 wt %-43 wt % of urethane dimethacrylate (UDMA).

2. The denture base resin for 3D printing according to claim 1, wherein the resin comprises 30.2 wt %-30.9 wt % of UDMA, and 0.5 wt %-2.6 wt % of diphenyl (2,4,6-trimethylbenzoyl) phosphine oxide (DTPO).

3. The denture base resin for 3D printing according to claim 1, wherein the resin comprises 30.2 wt %-30.9 wt % of UDMA, 0.5 wt %-2.6 wt % of DTPO, 0.0012 wt %-0.006 wt % of Erythrosin B, and 0.12 wt %-0.15 wt % of titanium dioxide (TiO₂).

4. The denture base resin for 3D printing according to claim 1, wherein the resin comprises 41.3 wt %-43 wt % of UDMA, and 0.4 wt %-4 wt % of diphenyl phosphine oxide (DTPO).

5. The denture base resin for 3D printing according to claim 1, wherein the resin comprises 41.3 wt %-43 wt % of UDMA, 0.4 wt %-4 wt % of DTPO as a photoinitiator, 0.0012 wt %-0.006 wt % of Erythrosin, and 0.12 wt %-0.15 wt % of TiO₂.

6. The denture base resin for 3D printing according to claim 1, wherein the resin comprises 1.6 wt %-2.1 wt % of ethyl 4-(dimethylaminomino) benzoic acid (DMAB).

7. The denture base resin for 3D printing according to claim 1, wherein the resin comprises 19.2 wt %-25 wt % of triethylene glycol dimethacrylate (TEGDMA).

8. The denture base resin for 3D printing according to claim 1, wherein the resin comprises 11.5 wt %-15 wt % of Bisphenol A glycidyl methacrylate (Bis-GMA).

9. The denture base resin for 3D printing according to claim 1, wherein the resin comprises 10 wt %-14.5 wt % of pentaerythritol tetraacrylate (PETRA).

10. The denture base resin for 3D printing according to claim 1, wherein the resin comprises 11.5 wt %-15 wt % of Di (trimethylolpropane)-tetraacrylate (Di-TMPTA).
